(19) United States
(12) Patent Application Publication
(10) Pub. No.: US 2016/0101246 A1
(43) Pub. Date: Apr. 14, 2016

(54) APPARATUS AND METHODS FOR PRODUCING AND DELIVERING A VAPOUR MEDICAMENT

(71) Applicant: Solaeromed Inc., Calgary (CA)

(72) Inventors: John H. Dennis, Calgary (CA); Ian G. Mcaffer, Calgary (CA); Stefan Dalberg, Calgary (CA); Francis Green, Calgary (CA); Andy Clark, Calgary (CA)

(73) Assignee: Solaeromed Inc., Calgary (CA)

(21) Appl. No.: 14/877,032

(22) Filed: Oct. 7, 2015

Related U.S. Application Data
(60) Provisional application No. 62/062,334, filed on Oct. 10, 2014.

(51) Int. Cl. A61M 11/02; A61M 11/04; A61M 15/00; A61K 31/02; A61M 11/00

(52) U.S. Cl. CPC A61M 11/02; A61K 31/02; A61M 11/003; A61M 15/009; A61M 11/04; A61M 2210/1035; A61M 2205/7536

(57) ABSTRACT

Disclosed are apparatus and methods for producing and delivering vapour medicament. An exemplary apparatus disclosed comprises: a gas inlet for receiving a gas from a source of pressurized gas; a chamber for receiving a liquid medicament therein, the chamber being in communication with the gas inlet for permitting entrance and expansion of the gas in the chamber in the presence of the liquid medicament to produce the vapour medicament; and a vapour outlet in communication with the chamber for delivering the vapour medicament.

### CROSS-REFERENCES TO RELATED APPLICATIONS

This application, claims the benefit of U.S. provisional patent application No. 62/062,334, filed on Oct. 10, 2014, the entirety of which is incorporated herein by reference.

### FIELD

The disclosure relates generally to apparatus and methods tor

delivery of medicament vapours, and more particularly to apparatus and methods for producing and delivering medicament in vapour form.

### BACKGROUND OF THE ART

Respiratory diseases such as asthma and chronic obstructive

pulmonary diseases (COPD) are characterized by the constriction or narrowing of the airways of the lungs. Asthma is a chronic disease in which sufferers have repeated attacks of difficulty in breathing and coughing. The prevalence and severity of asthma, as well as its associated mortality, have increased in the last few decades.

The majority of asthmatics (90-95%) suffer from the mild to moderate form of the disease and can control the disease with appropriate treatment. However, the 5-10% of asthmatics that suffer from the severe form of the disease, also known as severe persistent or acute severe asthma, are faced with frequent and sometimes life-threatening attacks. During an acute asthma attack, the airways (e.g., bronchi and bronchioles) are greatly narrowed by swelling (caused by inflammation of the inner lining of the bronchi/bronchioles), bronchoconstriction (caused by contraction of the bronchi/bronchioles smooth muscles) and mucus plugging (caused by the excess production of mucus produced by mucous gland secretions and cells lining the airway wall). The resultant narrowed airways make it more difficult for air to pass through and allow adequate gas exchange in the alveoli, the air sacs at the end of the bronchioles, resulting in reduction of oxygen (hypoxaemia) in the blood and reduced oxygen supply to vital organs. A severe asthma attack will require hospital admission and emergency care.

COPD is a slowly progressive disease of the airways that is characterized by the gradual loss of lung function. Patients with COPD often require emergency treatment and sometimes hospitalizations during periods of exacerbations of their disease. COPD leads to chronic airflow obstruction, which is defined as a persistent decrease in the rate of airflow through the lungs when the person breathes out (exhales). Symptoms such as wheezing and shortness of breath are relieved when airflow obstruction decreases by reversing bronchial smooth muscle spasm, inflammation, and increased secretions.

Cystic fibrosis is an example of an obstructive lung disorder that results in bronchiectasis and progressive declines in Forced Expiratory Volume in one second (FEV1). In this disease, there is a genetic defect in the transport of chloride or chlorine across the airway fining (epithelial) cells. This results in reduced water content in the mucous lining of the airways of the lung with increased viscosity of the mucus. The abnormal mucus becomes infected, which results in destruction and fibrosis of the bronchial wall. The mucous plugs and inflammation cause airway obstruction. Even with optimal treatment, most affected individuals die in their third or fourth decades.

Improvement in treatment of such conditions is desirable.

### SUMMARY

The disclosure describes components, apparatus and methods for producing and delivering vapours medicaments.

In one aspect, the disclosure describes an apparatus for producing and delivering a vapour medicament. The apparatus comprises:
a gas inlet for receiving a gas from a source of pressurized gas;
a chamber for receiving a liquid medicament therein, the chamber being in communication with the gas inlet for permitting entrance and expansion of the gas in the chamber in the presence of the liquid medicament to produce the vapour medicament;
a vapour outlet in communication with the chamber for delivering the vapour medicament; and
a barrier disposed upstream of the vapour outlet and configured to hinder passage of at least some of the liquid medicament through the vapour outlet and permit passage of at least some of the vapour medicament through the vapour outlet.

In another aspect, the disclosure describe a method for producing and delivering a vapour medicament The method comprises:
receiving a gas from a source of pressurized gas;
permitting expansion of the gas in the presence of a liquid medicament to produce the vapour medicament;
delivering at least, some of the vapour medicament through a vapour outlet while hindering delivery of at least some of the liquid medicament through the vapour outlet.

In another aspect, the disclosure describes an apparatus for producing and delivering a vapour medicament. The apparatus comprises:
a gas inlet for receiving a gas from a source of pressurized gas;
a chamber for receiving a liquid medicament therein, the chamber being in communication with the gas inlet for permitting entrance and expansion of the gas in the chamber in the presence of the liquid medicament to produce the vapour medicament;
a vapour outlet in communication with the chamber for delivering the vapour medicament; and
a liquid-absorbing member disposed inside of the chamber in a flow path between the gas inlet and the vapour outlet, the liquid-absorbing member being at least partially permeable to the gas.

In another aspect, the disclosure describes a method for producing and delivering a vapour medicament. The method comprises:
receiving a gas from a source of pressurized gas,
permitting expansion of the gas in the presence of a liquid medicament to produce the vapour medicament;
retaining at least some of the liquid medicament in a liquid-absorbing member while permitting passage of at least some of the gas through the liquid-absorbing member; and
delivering at least some of the vapour medicament through a vapour outlet.

In another aspect, the disclosure describes an apparatus for producing and delivering a vapour medicament. The apparatus comprises:
a chamber comprising a gas inlet for receiving a gas from a source of pressurized gas and a liquid inlet for receiving a liquid medicament from a liquid reservoir, the gas inlet and the liquid inlet being separate from each other, the chamber permitting expansion of the gas in the chamber in the presence of the liquid medicament to produce the vapour medicament; and
a vapour outlet in communication with the chamber for delivering the vapour medicament.

In another aspect, the disclosure describes a method for producing and delivering a vapour medicament. The method comprises:
receiving a liquid medicament into a chamber via a liquid inlet,
receiving a gas into the chamber via a gas inlet from a source of pressurized gas, the liquid inlet being separate from the gas inlet;
in the chamber, permitting expansion of the gas in the presence of the liquid medicament to produce the vapour medicament; and
delivering at least some of the vapour medicament through a vapour outlet.

In another aspect, the disclosure describes an apparatus for producing and delivering a vapour medicament. The apparatus comprises:
a gas inlet for receiving a gas from a pressurized gas reservoir;
a chamber for receiving a liquid medicament therein, the chamber being in communication with the gas inlet for permitting entrance and expansion of the gas in the chamber in the presence of the liquid medicament to produce the vapour medicament;
a vapour outlet in communication With the chamber for delivering the vapour medicament; and
a receptacle for receiving the pressurized gas reservoir, the receptacle having a longitudinal axis and the vapour outlet being disposed along the longitudinal axis of the receptacle.

In another aspect, the disclosure describes an apparatus for producing and delivering a vapour medicament. The apparatus comprises:

a storage reservoir containing both a liquid medicament and a pressurized gas;

a chamber comprising an inlet for receiving the liquid medicament and the gas from the storage reservoir, the chamber permitting expansion of the gas in the chamber in the presence of the liquid medicament to produce the vapour medicament; and

a vapour outlet in communication with the chamber for delivering the vapour medicament.

In another aspect, the disclosure describes a liquid medicament reservoir for use in an apparatus for producing and delivering a vapour medicament. The liquid medicament reservoir comprises:

a quantity of liquid medicament; and

an outer wall containing the liquid medicament, the outer wall comprising a frangible portion having a reduced wall thickness relative to the wall thickness of another portion of the outer wall.

Further details of these and other aspects of the subject matter of this application will be apparent from the detailed description and drawings included below.

### DESCRIPTION OF THE DRAWINGS

Reference is now made to the accompanying drawings, in which:

FIG. 1 shows a schematic view of an exemplary apparatus for producing and delivering a vapour medicament;

FIG. 2 shows a schematic view of another exemplary apparatus for producing and delivering a vapour medicament;

FIG. 3A shows a schematic view of another exemplary apparatus for producing and delivering a vapour medicament,

FIG. 3B shows a cress sectional view of an exemplary venturi channel in the vapour outlet of the apparatus of FIG. 3A;

FIG. 3C shows a cross sectional view of an exemplary liquid medicament reservoir;

FIG. 4A shows a top view of another exemplary apparatus for producing and delivering vapour medicament;

FIG. 48 shows a cross sectional view of the apparatus of FIG. 4A taken along line 4-4 of FIG. 4A;

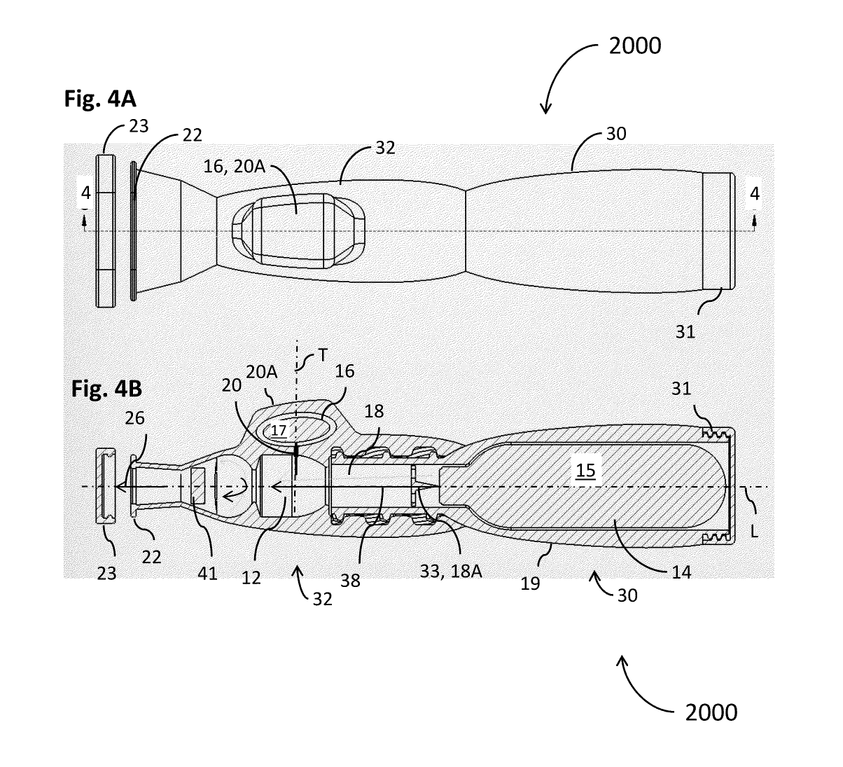

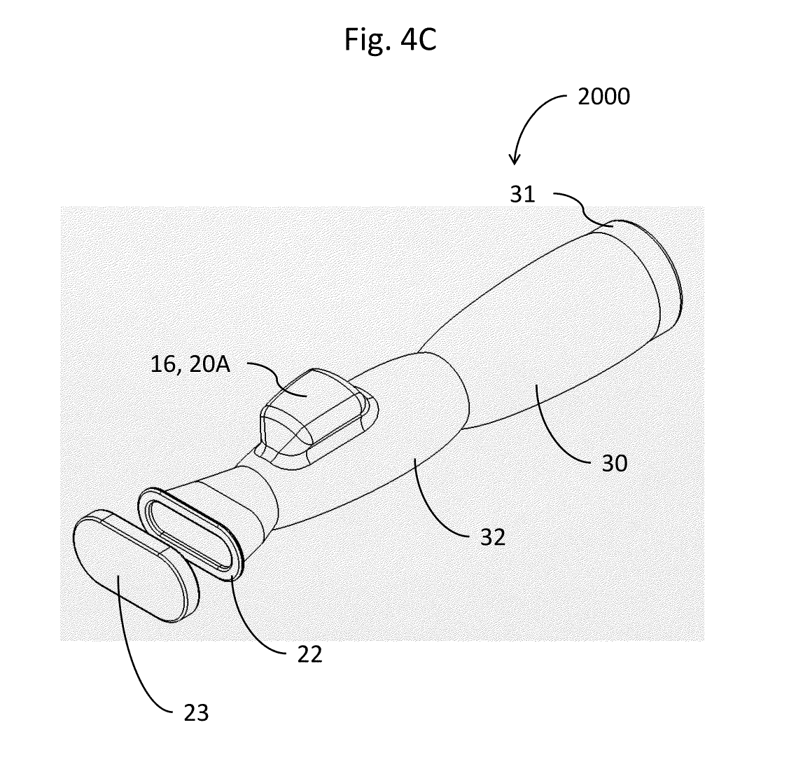

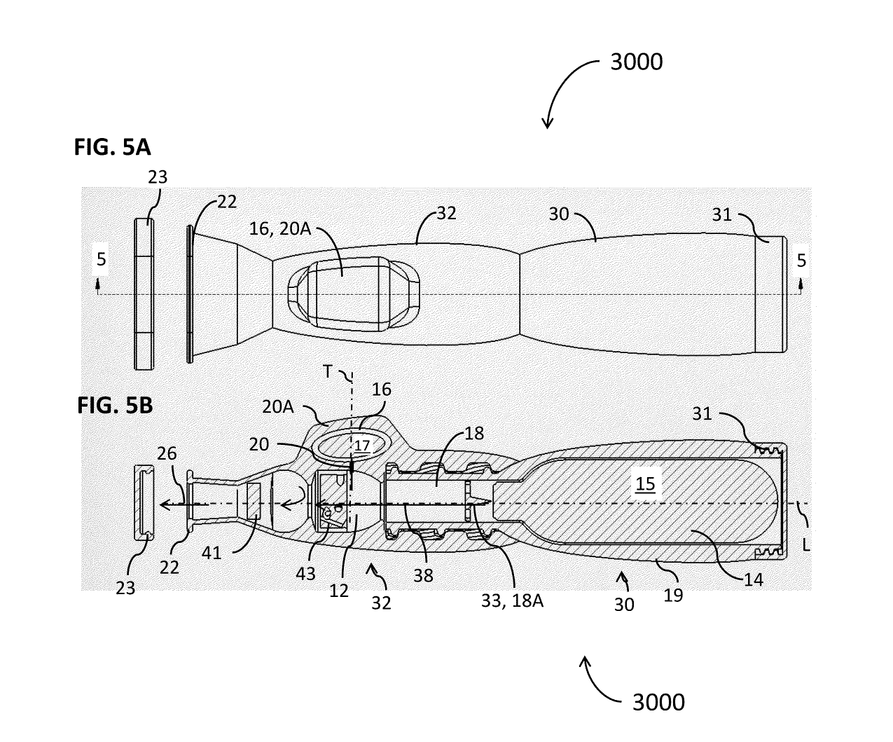

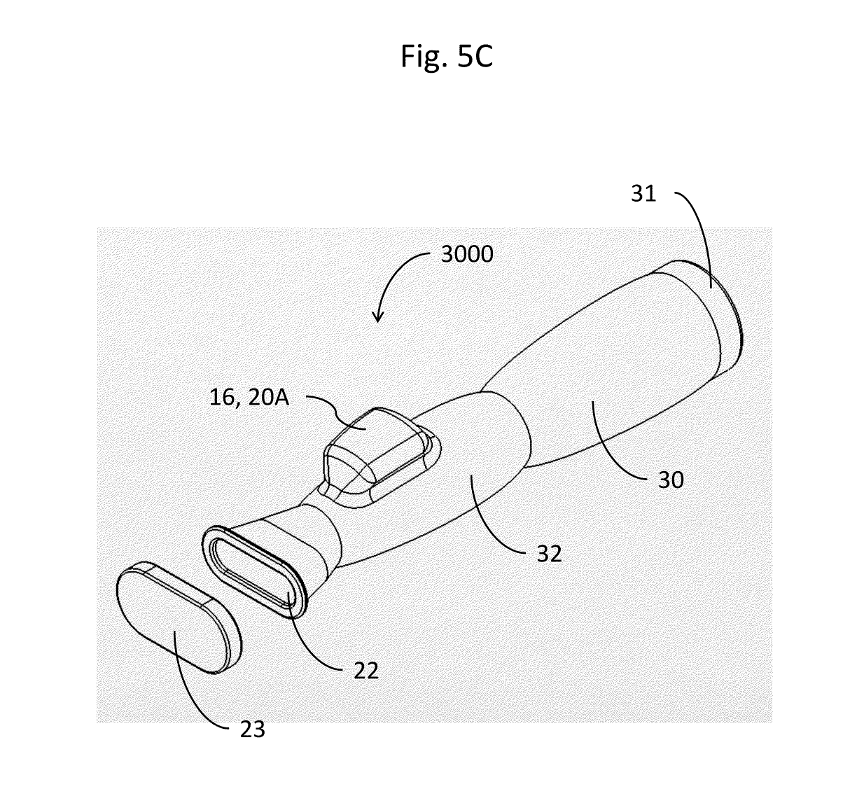

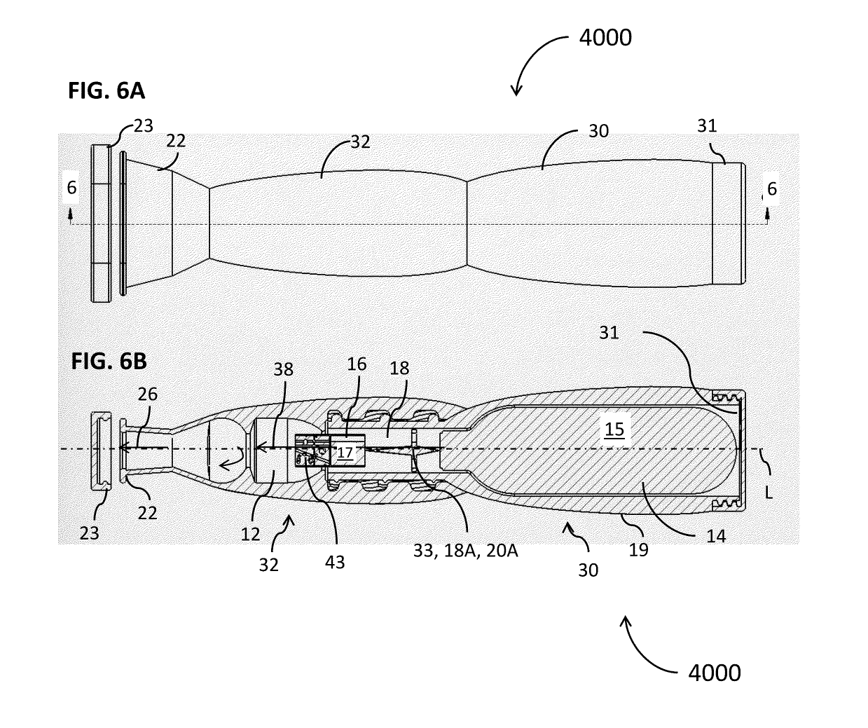

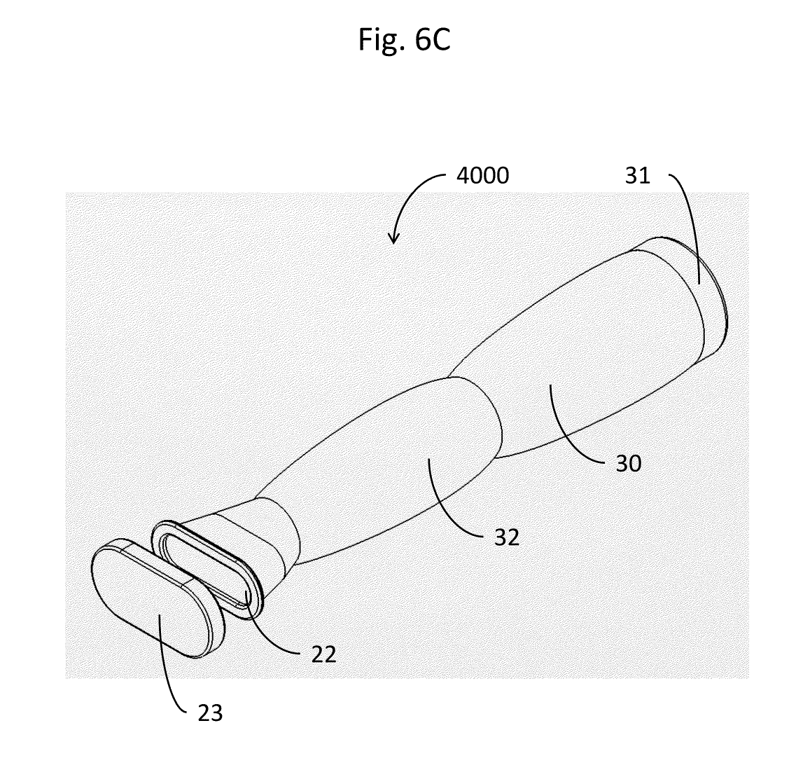

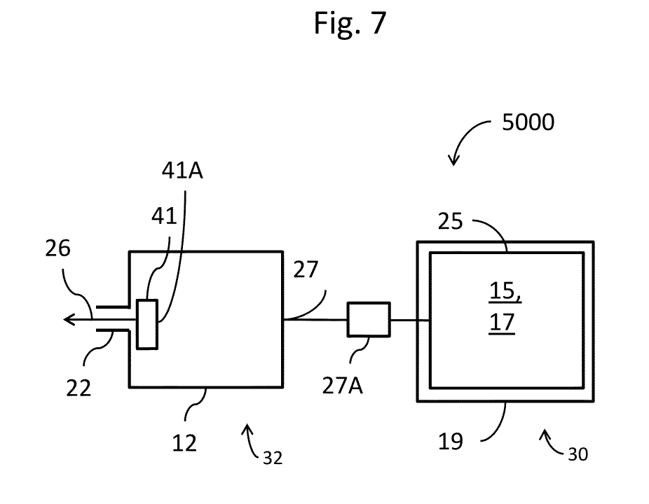

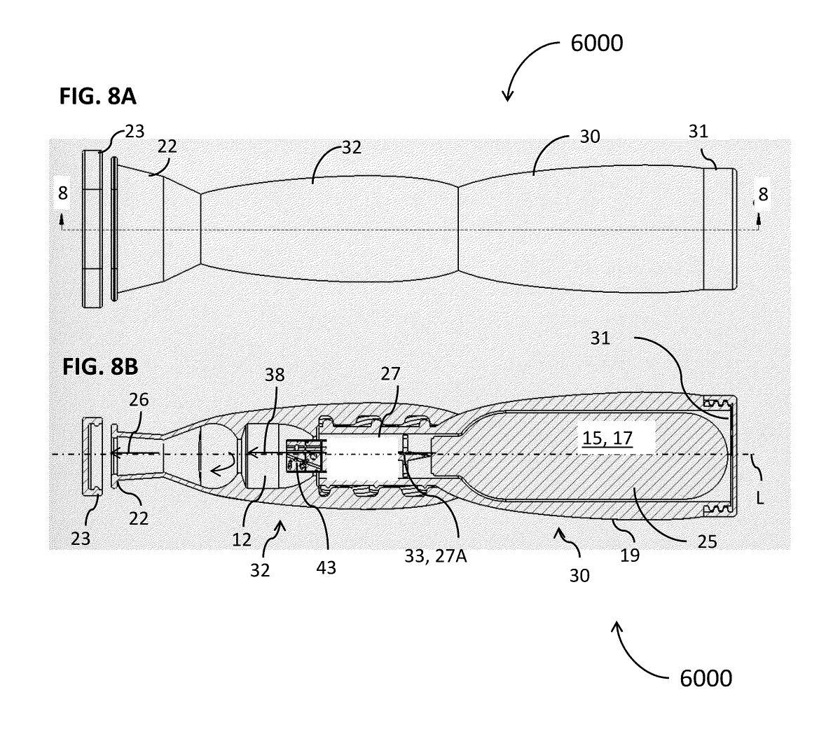

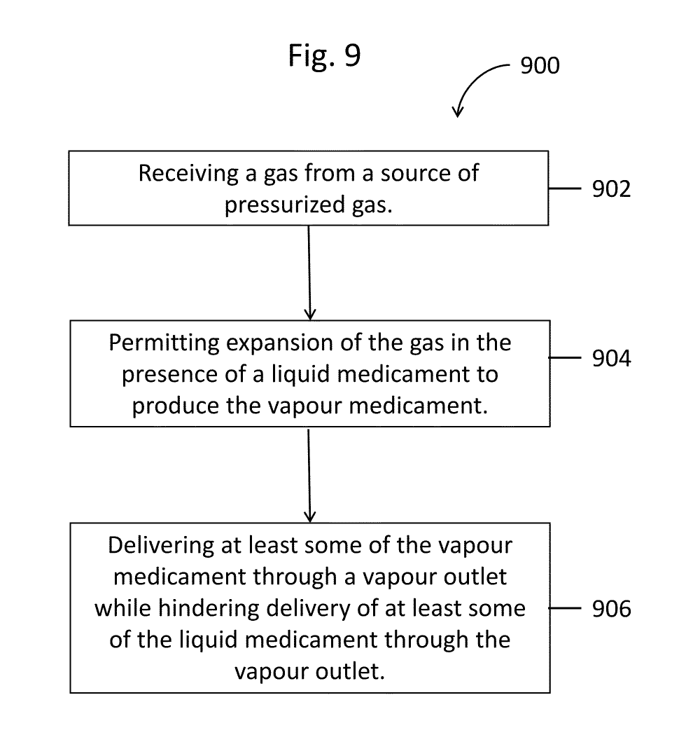

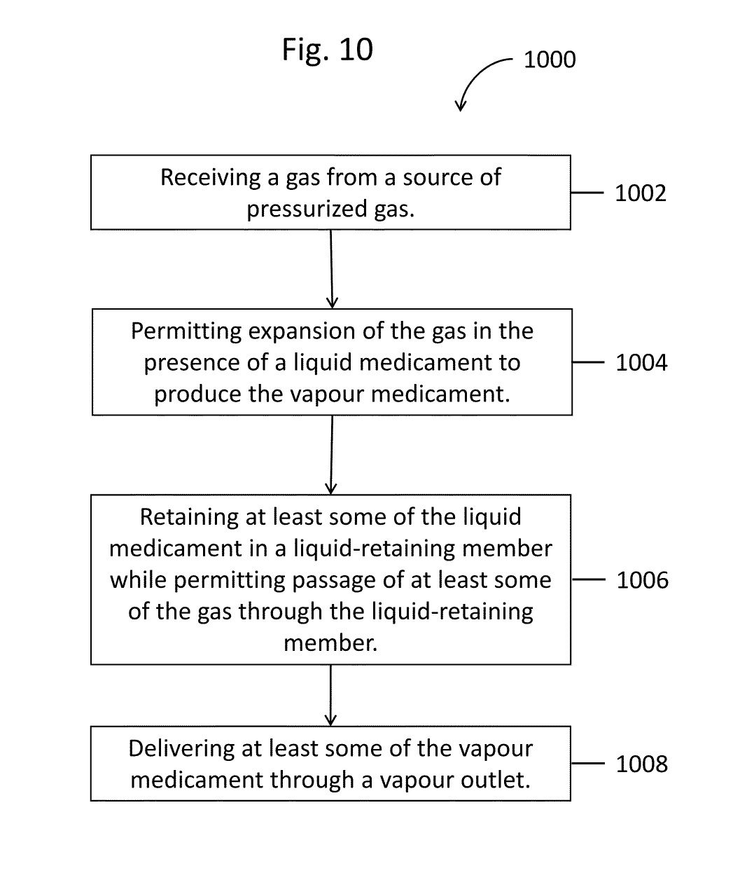

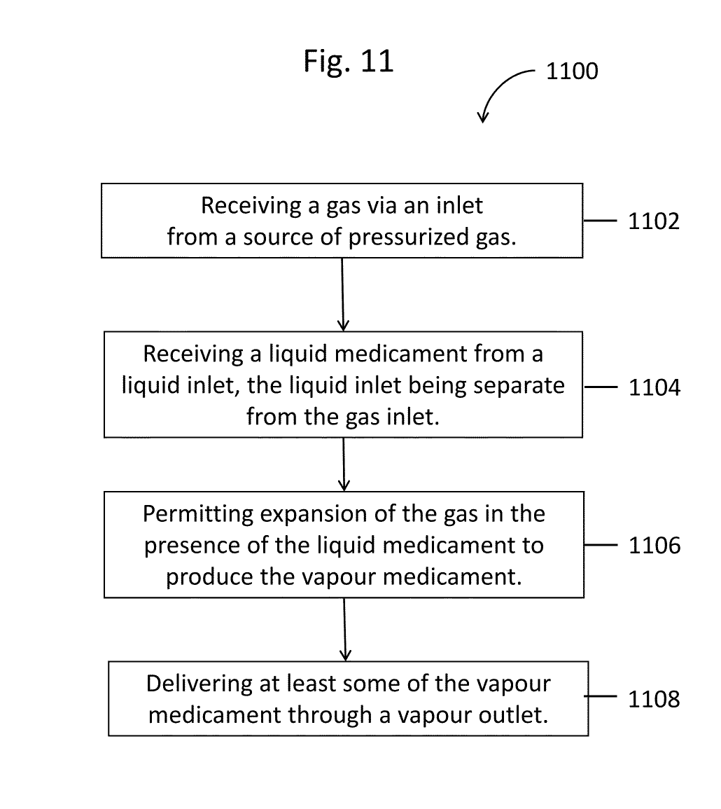

FIG. 4C shows an axonometric view of the apparatus of FIG. 4A;

FIG. 5A shows a fop view of another exemplary apparatus for producing and delivering vapour medicament;

FIG. 5B shows a cross sectional view of the apparatus of FIG. 5A taken along line 5-5 of FIG. 5A;

FIG. 5C shows an axonometric view of the apparatus of FIG. 5A;

FIG. 6A shows a top view of another exemplary apparatus for producing and delivering vapour medicament;

FIG. 6B shows a cross sectional view of the apparatus of FIG. 6A taken along line 6-6 of FIG. 6A;

FIG. 6C shows an axonometric view of the apparatus of FIG. 6A;

FIG. 7 shows a schematic view of another exemplary apparatus for producing and delivering a vapour medicament;

FIG. 8A shows a top view of another exemplary apparatus for producing and delivering vapour medicament;

FIG. 8B shows a cross sectional view of the apparatus of FIG. 8A taken along line 8-8 of FIG. 8A;

FIG. 9 shows a flowchart of an exemplary method of producing and delivering a vapour medicament,

FIG. 10 shows a flowchart of another exemplary method of producing and delivering a vapour medicament; and

FIG. 11 shows a flowchart of another exemplary method of producing and delivering a vapour medicament.

### DETAILED DESCRIPTION

Aspects of various embodiments are described through reference to the drawings.

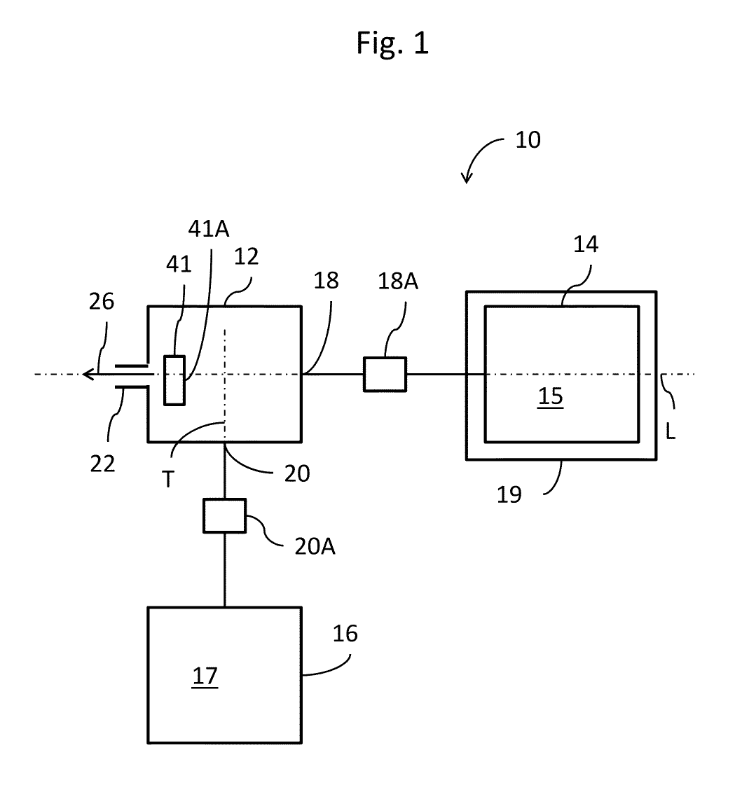

FIG. 1 schematically illustrates an exemplary apparatus 10 that may be used for producing and delivering vapour medicament to a subject (user) according to an embodiment of the present disclosure. The apparatus 10 may comprise a source of pressurized gas or reservoir 14 containing pressurized gas 15. Gas reservoir 14 may be in communication with chamber 12 via gas inlet 18. Apparatus 10 may also comprise liquid reservoir 16 containing liquid medicament 17. Liquid reservoir 18 may be in communication with chamber 12 via liquid inlet 20. Accordingly, chamber 12 may receive liquid medicament 17 and pressurized gas 15 via separate inlets and permit decompression (e.g. expansion) of gas 15 in the presence of liquid medicament 17 to produce vapour medicament 26. Accordingly, the production of vapour medicament 28 may be a result of the evaporation of the liquid medicament 17 in the presence of the expanding gas 15. Gas reservoir 14 may comprise a cartridge that may be removably received into or coupled to apparatus 10 via receptacle 13. Receptacle 19 may have longitudinal axis L.

Apparatus 10 may comprise vapour outlet 22 in communication with chamber 12 for delivering vapour medicament 26 from chamber 12 to a subject. The subject may be a mammal, for example a human, and delivery of vapour medicament 26 to the subject's lungs may occur upon inhalation of the vapour medicament 26 by the subject once it has been produced and delivered by the apparatus 10.

Delivery of vapour medicament 26 to the lungs of a subject may be achieved by inhalation. For example, vapour outlet 22 may be configured to interface with the mouth/lips of the subject and permit the subject to inhale vapour medicament 26. In some applications, vapour medicament 26 may permit a more effective and efficient delivery of the active ingredients of the medicament to the lungs. For example, liquid droplets from conventional aerosols may get intercepted in the mouth and/or throat of a subject and get swallowed by the subject instead of getting delivered to the lungs. However the use of a medicament in vapour form may permit a more efficient delivery to the lungs because the vapour form may permit more of the medicament to make it through the tortuous path between the mouth and the lungs of the subject.

In some embodiments, vapour outlet 22 may he disposed along longitudinal axis L of receptacle 19. For example, apparatus 10 may be configured such that gas inlet 18 and vapour outlet 22 are substantially coaxial. In some embodiments, gas inlet 18 and/or vapour outlet 22 may be disposed along axis L. Such arrangement may permit apparatus 10 to be relatively compact for ease of storage and portability. Liquid inlet 20 may be oriented substantially transversely to gas inlet 18. For example, liquid inlet 20 may have an axis T that is transverse (e.g., perpendicular) to the longitudinal axis L of receptacle 19.

Apparatus 10 may also comprise first activation mechanism 20A for causing liquid medicament 17 to enter chamber 12 and/or second activation mechanism 18A. for causing gas 15 to enter chamber 12. First activation mechanism 20A and second activation mechanism 18A may be actuatable independently of each other either manually by the subject, or may be actuatable independently automatically upon receiving a signal from the subject. First and/or second activation mechanisms 20A, 18A, may each comprise one or more valves or other means of causing liquid 17 and/or gas 15 to be delivered into chamber 12. Alternatively, first and/or second activation mechanisms 20A, 18A may be actuatable together simultaneously or otherwise by a single mechanism initiated by subject. First and/or second activation mechanisms 20A, 18A may be actuatable via buttons, switches, by selective rotation or compression of components of the apparatus 10, or by other means. First and second activation mechanisms 20A, 18A may permit the introduction of gas 15 and liquid medicament 17 either simultaneously or at different times. For example, liquid medicament 17 may be introduced into chamber 12 first in order to permit the expansion of gas 15 in chamber 12 and also in the presence of liquid medicament 17. First and second activation mechanisms 20A, 18A may permit a controlled introduction of gas 15 and/or liquid medicament 17 into chamber 12. For example, in some embodiments, second activation mechanism 18A may permit the flow rate of gas 15 to be controlled, stopped and/or restarted as desired.

Apparatus 10 may also comprise barrier 41 disposed upstream of the vapour outlet 22 and configured to hinder passage of at least some of liquid medicament 17 through the vapour outlet 22 and permit passage of at least some of vapour medicament 26 through vapour outlet 22 by filtering out droplets of liquid medicament 17 from a stream containing vapour medicament 26. Without being limiting, such barrier 41 may comprise one or more electrostatic surfaces 41A that partially obstruct a flow path to vapour outlet 22. Such electrostatic surface(s) 41A may attract liquid droplets that may be present in a stream containing vapour medicament 26 and substantially hinder them from exiting vapour outlet 22. Barrier 41 may provide an obstacle and cause a diversion in the flow path of vapour medicament 28 flowing out of vapour outlet 26 from chamber 12. Liquid medicament 17 hindered and/or captured by barrier 41 may be collected inside chamber 12 or other part of apparatus 10 and may be removed via the vapour outlet 22 if/when desired. Alternatively, the liquid medicament 17 captured by barrier 41 may remain inside chamber 12 or other part of apparatus 10 until it has been converted to vapour form by interaction with gas 15.

Gas reservoir 14 may comprise any suitable supply of pressurized gas 15 suitable for delivering gas 15 to chamber 12. For example, gas reservoir 14 may include a replaceable gas cartridge containing a gas that is suitable in size, shape and weight for use with a hand-held apparatus such as apparatus 10, or may alternatively be a connection to a larger source of gas 15 through which gas 15 may flow into the chamber 12 of the apparatus 10 via inlet 18.

Gas 15 contained or supplied by gas reservoir 14 may be suitable for at least non-continuous inhalation by the subject using the apparatus 10 and may, for example, comprise carbon dioxide (CO₂). Gas 15 may alternatively comprise a gas mixture comprising CO₂, oxygen and/or nitrogen (e.g., compressed air). In one embodiment, the concentration of CO₂ in gas 15 may be between about 2% by volume to about 20% per volume. In another exemplary embodiment, the concentration of CO₂ in gas 15 may be such that when the vapour medicament 26 is delivered to a subject's lungs, the concentration of CO₂ in the lungs is at least about 2%, and may be at least about 2% to about 20%, of the total lung capacity, wherein the total lung capacity comprises the fluid volume of the lung when fully inflated during normal breathing. Oxygen may also be present in amounts such that the concentration of oxygen may be at least about 15% to about 80% of a patient's total lung capacity, wherein the total lung capacity comprises the fluid volume of the lung when fully inflated during normal breathing, when vapour medicament 26 is delivered to the patient's lungs.

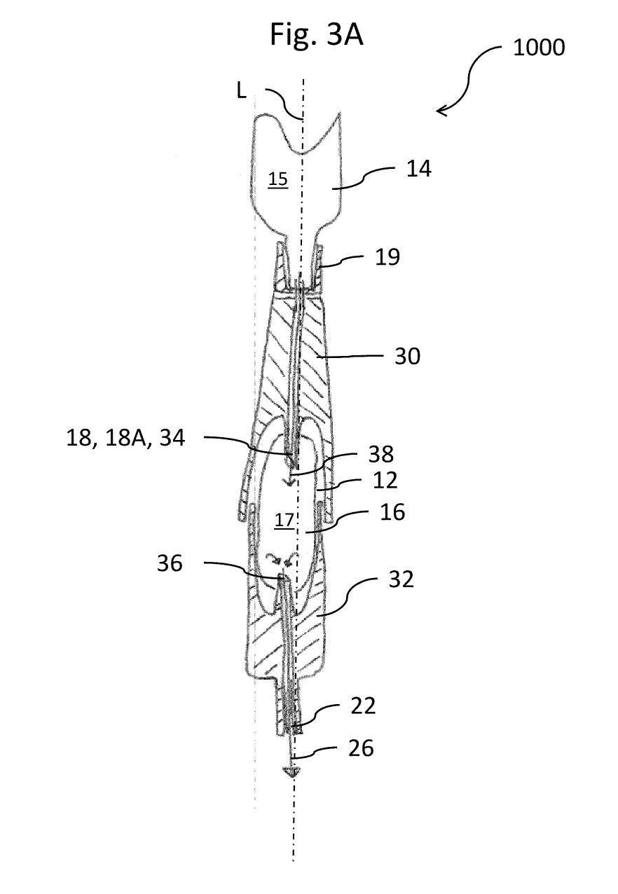

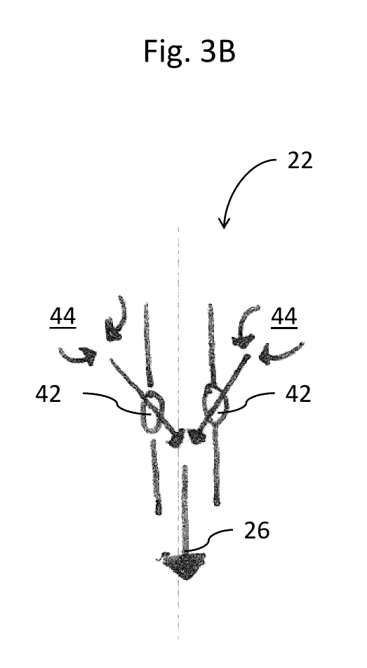

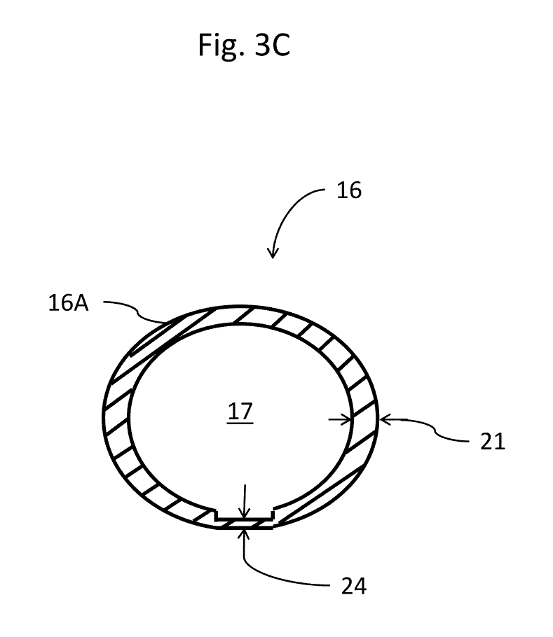

Gas 15 may also comprise substantially pure CO₂, a mixture comprising CO₂ and oxygen, or a mixture comprising CO₂, oxygen and nitrogen (e.g., compressed air). CO₂ may be a potent bronchial relaxant capable of opening up constricted airways within a short period of time after breathing in a gas mixture containing at least about 2% by volume CO₂. Other medicaments, for example bronchial relaxants containing safety and efficacy properties similar to that of CO₂, may also be suitable. Amounts of other gases may also be present in gas 15, for example those that have low viscosities, such as helium, and that other gases may be suitable for replacing oxygen and/or nitrogen. For example, gas 15 may comprise CO₂ and ambient air 44 (for example, as shown in the embodiment of outlet 22 shown in FIG. 3B). In various embodiments, apparatus 10 disclosed herein may permit a subject inhaling vapour medicament 26 delivered by the apparatus 10 to which a venturi opening 42 is added (FIG. 3B) to also inhale gas 15 which has entrained and been diluted by ambient air 44.

However, venturi opening 42 may also be placed at other locations on apparatus 10, for example and not intended to be limiting, on gas inlet 18, chamber 12, or generally any suitable location downstream of gas reservoir 14 wherein ambient air may be drawn into the flow of gas 15 as it moves from gas reservoir 14 towards and then from outlet 22.

Liquid medicament reservoir 16 may be any reservoir suitable for storing and supplying liquid medicament 17 to chamber 12. For example, reservoir 16 may be a replaceable reservoir suitable in size, shape and weight for use with a hand-held apparatus such as apparatus 10. For example, reservoir 16 may be a capsule, container or cartridge that is replaceably inserted into apparatus 10, or may be a container or capsule that is permanently installed in the apparatus 10 and that contains liquid medicament 17. Alternatively, reservoir 16 may comprise a connection to a relatively larger source of liquid medicament 17 for supplying liquid medicament 17 to chamber 12 via liquid medicament inlet 20.

Liquid medicament 17 may comprise a liquid containing one or more active pharmaceutical ingredients. For example, liquid medicament 17 may comprise a bronchial relaxant or a liquid drug capable of opening up constricted airways of a subject within a short period of time after being delivered to the lungs of the subject. Alternatively, liquid medicament 17 may comprise any suitable drug in liquid form that may be administered to a subject in vapour form. For example, liquid medicament 17 may be a perfluorocarbon that, without being bound to theory, is capable of dissolving large quantities of gasses, including CO₂. Alternatively, liquid medicament 17 may be a combination of more than one perfluorocarbon. Without being limited, examples of suitable perfluorocarbons include perfluoroalkanes, perfluoroethers, and perfluoro amines, or more specifically perfluorodecalin, perfluorohexane, octafluoropropane, perfluoroperhydrophenanthrene, perfluorobutane, perfluorooctane, perfluoromethyldecalin, perfluorocarbons containing bromide such as perfluorooctylbromide, perfluorodecalin, perfluorooctylethane, bis(perfluorobutyl)ethane or using the trade names, such as FC-43, FC-40, FC-5312, FC-77, FC-75 (3M Co), Rimar 101 (Mitsubishi, Milan) and Caroxin. It is understood that liquid medicament 17 of the present disclosure may comprise a mixture of two, three, four or more compatible perfluorocarbons.

In some exemplary embodiments, the concentration of perfluorocarbon may be such that when the perfluorocarbon is inhaled by a subject, the concentration of perfluorocarbon in the subject's lungs when the lungs are fully inflated during normal breathing may be between about 1 mg/liter and about 500 mg/liter.

As used herein, a “vapour medicament”, or “vapour”, refers to a gaseous state of a medicinal substance, and includes, for example and without being limited, liquid medicaments evaporated form or otherwise converted into gaseous form, for example, by vaporization of the liquid. Without being limiting, vapour medicaments may be gaseous forms of active pharmaceutical liquid ingredients, including liquid medicaments and/or bronchial relaxant liquids, for example one or more perfluorocarbons, as described above.

In use of apparatus 10, a subject may initiate release of gas 15 into chamber 12 through gas inlet 18, for example and without being limiting, through activation mechanism 18A for the release of gas 15. Release of gas 15 may entrain liquid medicament 17 into chamber 12 though liquid medicament inlet 20, or, alternatively some of liquid medicament 17 may already be present in chamber 12 (e.g., pre-stored in chamber 12 or previously injected into chamber 12 via activation mechanism 20A). The increase in volume and the reduction in pressure afforded by chamber 12 compared to gas reservoir 14, and the presence in chamber 12 of liquid medicament 17 supplied by liquid medicament reservoir 16 may permit decompression (e.g. expansion) of gas 15 as it enters chamber 12 to produce a vapour medicament 26 from liquid medicament 17.

Vapour medicament 26 may be delivered from chamber 12 by the release of gas 15 from gas reservoir 14 and through vapour outlet 22. Delivery of vapour medicament 26 to the lungs of the subject may occur upon inhalation by the subject as apparatus 10 delivers vapour medicament 26 via vapour outlet 22.

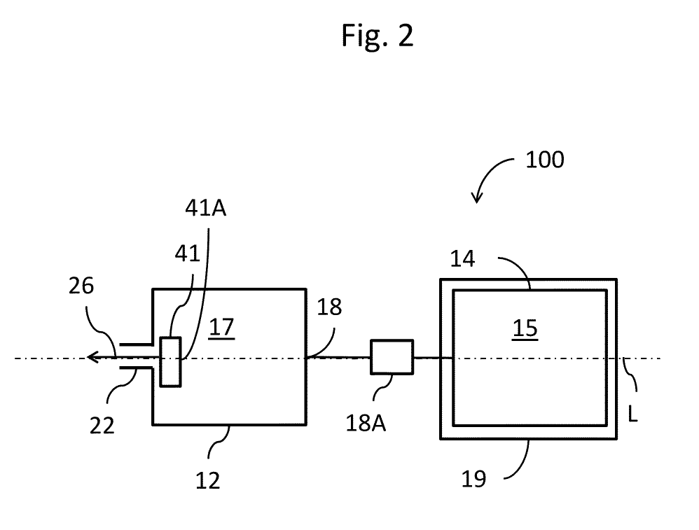

Referring now to FIG. 2, another exemplary apparatus 100 for producing and delivering vapour medicament 26 is schematically shown. Apparatus 100 may comprise one or more components described above in relation to apparatus 10 and therefore description of such components has not been repeated below. The apparatus 100 may comprise gas reservoir 14 in communication with inlet 18 for supplying gas 15 to chamber 12, which may contain liquid medicament 17 pre-stored therein. Chamber 12 may permit entrance of gas 15 via gas inlet 18 and decompression (e.g., expansion) of gas 15 in the presence of liquid medicament 17 to produce vapour medicament 26. In some embodiments, liquid medicament 17 may be pre-stored in chamber 12 prior to the release of gas 15 into chamber 12. Chamber 12 may be connected to gas reservoir 14 via inlet 18, enabling chamber 12 to receive gas 15. Apparatus 100 may comprise vapour outlet 22 for delivering vapour medicament 26 from chamber 12. Apparatus 100 may comprise a vapour outlet 22 in communication with chamber 12 for delivering the vapour medicament 26 from the chamber 12. Apparatus 100 may also comprise activation mechanism 18A for causing the gas 15 to enter the chamber 12.

Apparatus 100 may also comprise barrier 41 disposed upstream of the vapour outlet 22 and configured to hinder passage of at least some of liquid medicament 17 through vapour outlet 22 and permit passage of at least some vapour medicament 26 through the vapour outlet 22. Apparatus 100 may be configured such that gas inlet 18 and vapour outlet 22 may be substantially coaxial.

Referring now to FIG. 3A, another exemplary apparatus 1000 for producing and delivering vapour medicament 26 is shown. Apparatus 1000 may comprise one or more components already described above and description of such components has not been repeated below. Apparatus 1000 may comprise gas reservoir 14 for supplying gas 15 and chamber 12 comprising liquid medicament reservoir 16 for supplying liquid medicament 17, wherein gas reservoir 14 may be in communication with inlet 18, and wherein chamber 12 permits entrance of gas 15 from gas reservoir 14 via inlet 18 and permits decompression (e.g., expansion) of gas 15 in the presence of liquid medicament 17, producing vapour medicament 26. Apparatus 1000 may comprise receptacle 19 for receiving the gas reservoir 14 for facilitating coupling of gas reservoir 14 to gas inlet 18. Receptacle 19 may have longitudinal axis L. Vapour outlet 22 may be disposed along longitudinal axis L of receptacle 19 and in communication with chamber 12.

Apparatus 1000 may be configured such that gas inlet 18 and vapour outlet 22 are substantially coaxial. In some embodiments, liquid medicament 17 may be pre-stored in chamber 12 prior to the release of gas 15 into chamber 12.

Chamber 12 may comprise a pierceable/frangible capsule installed in the apparatus 1000 and containing the liquid medicament 17. Apparatus 1000 may comprise first body 32 defining at least part of chamber 12 and second body 30 defining at least part of receptacle 19. The first body 32 and second body 30 may be configured to be engaged to each other, for example, by being threadably engaged. Second body 30 and first body 32 may alternatively be removeably secured, for example, by friction, snap or other type of fitment.

In some embodiments, gas inlet 18 may comprise a piercing and optionally non-coring (e.g., “Huber”) needle 34. Upon bringing first body 32 and second body 30 together, such needle 34 may pierce reservoir 14 and allow gas 15 to flow info chamber 12 comprising the liquid medicament 17. In some embodiments, vapour outlet 22 may also comprise a piercing and optionally non-coring (e.g., “Huber”) needle 36. Accordingly, bringing first body 32 and second body 30 together may cause such needle 36 to pierce the chamber 12. Upon being pierced and secured between second body 32 and first body 30, chamber 12 may be made to be in fluid communication with gas reservoir 14 and vapour outlet 22 and may enable the delivery of vapour medicament 26 produced in chamber 12.

In some embodiments, first body 32 and second body 30 may, for example, be permanently attached, integrally formed, or may not allow movement toward each other. In such embodiments, apparatus 1000 may further comprise a activation mechanism, for example comprising a spring loading mechanism, which may be actuated automatically or manually by the user, and which may facilitate piercing of the chamber 12 by needles 34 and 36, enabling chamber 12 to be made to be in fluid communication with gas reservoir 14 and vapour outlet 22, and therefore may enable the delivery of vapour medicament 26 through outlet 22.

Needles 34 and 36 may be hollow to facilitate gas 15 to flow info chamber 12 from reservoir 14. Alternatively, and without being limiting, needles 34 and 36 and may retracted by the activation mechanism upon piercing of chamber 12 to enable the flow of gas through chamber 12. The flow rate of gas 15 may therefore be controlled or modulated by the thickness of the needles 34 and 36, the extent of retraction of needles 34 and 36, or where appropriate, the diameter of the orifice of optionally hollow needles 34 and 36. In some embodiments, apparatus 1000 may further comprise a flow regulator to regulate and/or control the rate at which gas is released into chamber 12.

In reference to FIG. 3B, the apparatus disclosed herein may optionally comprise one or more venturi openings 42 to enable ambient air 44 to be drawn into and mixed with the flow of vapour medicament 26 to further dilute the concentration of COs or other active ingredients in vapour medicament 26 for delivery to the lungs of the subject. Without being limiting, venturi openings 42 may be included on the vapour outlet 22, as depicted in FIG. 3B. The use of venturi openings 42 may enable higher concentrations of gas 15 to be supplied by gas reservoir 14 and/or higher concentrations of liquid medicament 17 to be supplied by liquid medicament reservoir 16 and still achieve the aforementioned concentration of each in the subject's lungs upon inhalation by the subject by entrapment of ambient air 44 and therefore dilution. This may allow for repeated uses of each of gas reservoir 14 and reservoir 16, and/or a smaller, more portable size and shape for the apparatus 1000. Such a configuration may be useful for patients requiring unexpected or emergency use of the apparatus and who may require a relatively compact and portable means that facilitates the inhalation of vapour medicament 26 when needed.

As described above with respect to apparatus 10, venturi openings 42 may also be placed at other locations of apparatus 1000. For example, and without being limiting, venturi openings 42 may be placed on one or more of gas inlet 18, chamber 12, second body 30, first body 32 or generally any suitable location downstream of gas reservoir 14 wherein ambient air may be drawn into the flow of gas 15 as it flows from gas reservoir 15 to or from outlet 22.

As shown in FIG. 3C, in some embodiments, liquid medicament reservoir 18 may comprise an outer wall 16A for containing the liquid medicament 17 therein. The outer wall 16A may comprise a first region having a first wall thickness 21 and a second region having a second wall thickness 24. The first wall thickness 21 and second wall thickness 24 may be the same or different. In some embodiments of liquid medicament reservoir 16 where the second thickness 24 is less than first thickness 21, the differential in thickness may still provide the requisite structural strength to the liquid medicament reservoir 16 to ensure the liquid medicament 17 remains safety in the reservoir 16 during storage of the apparatus, and may also reduce the risk of accidental or unwanted discharges of the liquid medicament 17. However, the region having a lesser second thickness 24 may also facilitate greater ease of use of the apparatus by reducing the necessary pressure required for a user to manually initiate release of liquid medicament 17 from liquid medicament reservoir 16, or alternatively to reduce the force or energy required to automatically do so. When used in conjunction with apparatus 1000, this exemplary embodiment of liquid medicament reservoir 16 may be pierced with needle 34 in the region having first thickness 21, permitting the pressure created by the release of gas from reservoir 14 to bear down on the region having the second thickness 24, which would enable needle 36 to pierce chamber 12, and may also enable chamber 12 to be brought in fluid communication with gas reservoir 14 and vapour outlet 22. Accordingly, the portion of the outer wall 16A having the reduced wall thickness may function as a frangible portion, in various embodiments, the second thickness 24 may be up to about 25% of the first thickness 21. In some embodiments, the second thickness 24 may be up to about 50% of the first thickness 21. In other embodiments, the second thickness 24 may be up to about 75% of the first thickness 21. In various embodiments, the outer wall 16A may have a wall thickness of about 0.05 mm to about 2 mm.

In operation, apparatus 1000 may be assembled as described above. Upon second body 30 and first body 32 being coupled and upon release of gas 15, gas 15 supplied by the gas reservoir 14 may flow into chamber 12 via inlet 18. Alternatively, the puncturing device(s) may be automatically or manually activated, facilitating the piercing of chamber 12 to release gas 15 into chamber 12 via inlet 18. This may be achieved through the use of buttons, sensors, switches, knobs or a combination of buttons, sensors, switches and/or knobs located on or in apparatus 1000. For example, the puncturing device may be activated upon a sensor sensing that the subject is placing their mouth on or in proximity to outlet 22.

The increase in volume afforded by the chamber 12, and therefore the reduction in pressure compared to the gas reservoir 14, and the presence in the chamber 12 of the liquid medicament 17 supplied by liquid medicament reservoir 18 may permit decompression (e.g., expansion) of gas 15 in chamber 12 to produce vapour medicament 28.

The vapour medicament 26 may be delivered from the chamber 12 to vapour outlet 22 and be driven by the flow of gas 15 from the gas reservoir 14.

Referring now to FIGS. 4A-4C, there is shown another exemplary apparatus 2000 for producing and delivering vapour medicament 28. Apparatus 2000 may comprise one or more components already described above and description of such components has not been repeated below. Apparatus 2000 may comprise gas reservoir 14 for supplying gas 15 and liquid medicament reservoir 18 for supplying liquid medicament 17, each of which may be in communication with chamber 12 via inlets 18 and 20 respectively. Chamber 12 may permit entrance of gas 15 from gas reservoir 14 via gas inlet 18 and entrance of liquid medicament 17 from liquid medicament reservoir 16 via liquid medicament inlet 20, permitting decompression (e.g., expansion) of gas 15 in the presence of liquid medicament 17 and thereby producing vapour medicament 26. The apparatus 2000 may comprise a vapour outlet 22 in communication with chamber 12 for delivering vapour medicament 26 from chamber 12. In some embodiments, liquid medicament 17 may be delivered to chamber 12 prior to the release of gas 15 into chamber 12.

Gas reservoir 14 may be received in receptacle 19 and enclosed therein via distal portion cap 31. Gas reservoir 14 may, for example, be a commercially available and pierceable pressurized gas reservoir or cartridge. Gas reservoir 14 and/or receptacle 19 may have longitudinal axis L. Vapour outlet 22 may be disposed along longitudinal axis L of receptacle 19. Liquid inlet 20 may be oriented substantially transversely to gas inlet 18. For example, liquid inlet 20 may nave an axis T that is transverse (e.g., perpendicular) to longitudinal axis L of receptacle 19. Apparatus 2000 may be configured such that gas inlet 18 and vapour outlet 22 are substantially coaxial so that the flow of gas 15 from gas reservoir 14 to vapour outlet 22 may be substantially linear except for any diversion(s) that may be caused by barrier 41.

Apparatus 2000 may comprise first body 32 defining at least part of chamber 12 and second body 30 defining at least part of receptacle 19. First body 32 and second body 30 may be configured to be engaged to each other, for example, by being threadably engaged.

Apparatus 2000 may also comprise first activation mechanism 20A for causing liquid medicament 17 to enter chamber 12 and/or second activation mechanism 13A for causing gas 15 to enter chamber 12. Fitment of first body 32 and second body 30 may be configured to enable a subject to bring second body 30 and first body 32 toward each other in order to allow puncture device 33 to puncture gas reservoir 14 and cause the release of gas 15 from gas reservoir 14 using, for example, one or more tips. Accordingly, second activation mechanism 18A may comprise puncture device 33. Gas 15 may be directed from gas reservoir 14 into the chamber 12 along flow path 38. Puncture device 33 may be fixed to second body 30 or first body 32.

In some embodiments, first body 32 and second body 30 may, for example, be permanently attached, integrally formed, or may not allow movement toward each other. In such embodiments, puncture device 33 may comprise, for example, a spring loaded mechanism incorporating one or more tips, which may be actuated automatically or manually by the user through, for example, the use of buttons, switches, knobs and/or sensors, and which may allow puncture device 33 to puncture gas reservoir 14 and cause the release of gas 15 from gas reservoir 14. The tip of puncturing device 33 may be hollow to facilitate gas 15 to flow therethrough into chamber 12. Alternatively, and without being limiting, tip may be retracted by the puncture device 33 upon piercing of reservoir 14 that may enable the flow of gas to chamber 12. The flow rate of gas 15 may therefore be controlled or modulated by the width or thickness of the tip, the extent of retraction of the tips, or for hollow tips, the diameter of the orifice of the tip. In some embodiments, apparatus 2000 may further comprise a flow regulator to regulate and/or control the rate at which gas is released into chamber 12.

First activation mechanism 20A may be user-actuatable. For example liquid medicament reservoir 16 may comprise a compressible pouch/capsule that may be depressed by a subject in order to force liquid medicament 17 from reservoir 16 to chamber 12 via liquid inlet 20.

Apparatus 2000 may also comprise barrier 41, as described above, and outlet cap 23 to cover vapour outlet 22 when not in use.

FIG. 4C depicts an axonometric view of apparatus 2000. showing the first body 32, second body 30, distal portion cap 31, outlet 22, outlet cap 23, liquid medicament reservoir 16 and first activation mechanism 20A.

In operation, apparatus 2000 may function such that, upon second body 30 and first body 32 being brought together and secured and upon release of gas 15 by actuation of second activation mechanism 18A, gas 15 supplied by the gas reservoir 14 may flow into chamber 12 via inlet 18. Either simultaneously or prior to release of gas 15, liquid medicament 17 may he released from the reservoir 16 upon subject's application of pressure to reservoir 16 (e.g., compressible pouch) to deliver liquid medicament 17 into the chamber 12 via inlet 20. Alternatively, puncture device 33 may be automatically or manually activated by other means to facilitate the release gas 15 into chamber 12 via inlet 18. This may be achieved through the use of buttons, sensors, switches, knobs or a combination of buttons, sensors, switches and/or knobs located on or in apparatus 2000. For example, the puncturing device 33 may be activated upon a sensor sensing that the subject is placing their mouth on or in proximity to outlet 22. Said buttons and/or sensors may be placed on any location on the surface of the apparatus 2000 to enable ease of access for the user.

The increase in volume afforded by chamber 12, and the reduced pressure compared to gas reservoir 14, and the presence in the chamber 12 of liquid medicament 17 supplied by subject applying pressure to liquid medicament reservoir 16 via first activation mechanism 20A, may permit decompression (e.g. expansion) of gas 15 as it enters chamber 12 to produce a vapour medicament 26 of the liquid medicament 17.

Barrier 41 may also remove (e.g., filter out) droplets of the liquid medicament 17 from the vapour medicament 26 before delivery out of the apparatus 2000.

Referring now to FIGS. 5A-5C, there is shown another exemplary apparatus 3000 for producing and delivering a vapour medicament 26. Apparatus 3000 may comprise one or more components already described above and description of such components has not been repeated below. FIG. 5B shows apparatus 3000 comprising gas reservoir 14 for supplying gas 15 and liquid medicament reservoir 16 for supplying liquid medicament 17, each of which may be in communication with chamber 12 via inlets 18 and 20 respectively. Apparatus 3000 may comprise liquid-absorbing member 43 disposed inside of chamber 12 between gas inlet 18 and vapour outlet 22. Liquid-absorbing member 43 may be at least partially permeable to the gas 15. For example, liquid-absorbing member 43 may have one or more passages therethrough for permitting the flow of gas 15. Vapour outlet 22 may be in communication with chamber 12.

Liquid-absorbing member 43 may comprise a porous material, for example, a sponge, wherein the internal porous surface area of the liquid-absorbing member 43 is significantly greater than the outer surface. Liquid-absorbing member 43 may comprise an absorbent material which may absorb (e.g., retain) some of liquid medicament 17 but may also be at least partially permeable to the flow of gas 15 therethrough. The passing of gas 15 through liquid-absorbing member 43 and also the expansion of gas 15 inside of chamber 12 may cause the production of vapour medicament 26 inside of chamber 12. Liquid-absorbing member 43 may promote the production of vapour medicament 26 by providing a relatively large effective surface area of liquid medicament 17 during its interaction with gas 15. Vapour medicament 26 produced may be delivered from chamber 12 via vapour outlet 22.

FIG. 5C depicts an axonometric view of apparatus 3000, showing the first body 32, second body 30, distal portion cap 31, outlet 22, outlet cap 23, liquid medicament reservoir 16 and first activation mechanism 20A.

Referring now to FIGS. 6A-6C, there is shown another exemplary apparatus 4000 for producing and delivering vapour medicament 26. Apparatus 4000 may comprise one or more components already described above and description of such components has not been repeated below. Apparatus 4000 may comprise gas reservoir 14 in communication with chamber 12 via outlet 18 and liquid medicament reservoir 16 for supplying liquid medicament 17. Liquid medicament reservoir 16 may be disposed in flow path 38 and upstream of chamber 12. Apparatus 3000 may comprise liquid-absorbing member 43 as described above disposed inside of chamber 12 between gas inlet 18 and vapour outlet 22.

Apparatus 4000 may also comprise first activation mechanism 20A for causing the liquid medicament 17 to enter the chamber 12 and/or second activation mechanism 18A for causing gas 15 to enter chamber 12. First and second activation mechanisms 20A and 18A may both comprise puncture device 33, which may have two opposing piercing tips. For example apparatus 4000 may comprise a pierceable liquid medicament reservoir 16 that is punctured (and may be pierced fully through, creating a flow passage for gas therethrough) by a first piercing tip of puncture device 33 when first body 32 and second body 30 are brought together via thread-able or other engagement, or upon activation of puncture device 33 manually or automatically by user by means described above. Puncture device 33 may be on or in first body 32 or second body 30.

The puncturing of liquid medicament reservoir 16 and also of gas reservoir 15 may cause gas 15 exiting gas reservoir 14 to force liquid medicament 17 into liquid-absorbing member 43. As explained above in relation to apparatus 3000, the flow of gas 15 through liquid-absorbing member 43 and expansion of gas 15 in the presence of liquid medicament 17 may cause vapour medicament 26 to be produced in chamber 12 and delivered via vapour outlet 22.

FIG. 6C depicts an axonometric view of apparatus 4000, showing the first body 32, second body 30, distal portion cap 31, outlet 22, and outlet cap 23.

In operation of apparatus 4000, upon second body 30 and first body 32 being brought together by longitudinal movement of the two bodies 30, 32, gas 15 released from gas reservoir 14 may flow into inlet 18 and be driven into liquid medicament reservoir 18 punctured by the piercing member 33 and also through liquid-absorbing member 43. Alternatively, puncturing device 33 may be automatically or manually activated by other means to facilitate the release gas 15 into chamber 12 via inlet 18, for example, as described above.

Liquid medicament 17 may be forced into liquid-absorbing member 43 by virtue of the flow of gas 15 induced by a differential in pressure between the gas reservoir 14 and the chamber 12. The flow of gas 15 through liquid-absorbing member 43 and the expansion of the gas 15 in the presence of liquid medicament 17 may cause vapour medicament 26 to be produced due to evaporation of liquid medicament 17.

Vapour medicament 25 and gas 15 may continue to be delivered from the chamber 12 via vapour outlet 22.

Referring now to FIG. 7, there is shown a schematic illustration of an exemplary apparatus 5000 that may be used for producing and delivering vapour medicament to a subject (user) according to an embodiment of the present disclosure. Apparatus 5000 may comprise one or more components already described above and description of such components has not been repeated below. FIG. 7 shows apparatus 5000 may comprise a pressurized storage reservoir 25, for example a cylinder, comprising both gas 15 and liquid medicament 17, and which may be in communication with chamber 12 via inlet 27. Apparatus 5000 may also comprise activation mechanism 27A for causing gas 15 and liquid medicament 17 to be released from storage reservoir 25 and to enter chamber 12, and which may be manually or automatically activated by user. Apparatus 5000 may comprise barrier 41 and electrostatic surface 41A.

In operation, apparatus 5000 may be activated by engagement of the first 32 and second body 30, or by user manually or automatically to release gas 15 and liquid medicament 17 simultaneously into chamber 12 as described above in relation to apparatus 2000, wherein release of the pressurized gas 15 and liquid medicament 17 from reservoir 25 may enable expansion of the gas 15 in the presence of liquid medicament 17 in chamber 12, and may cause vapour medicament 26 to be produced. Vapour medicament 26 may continue to be delivered from the chamber 12 via vapour outlet 22.

Referring now to FIGS. 8A-8B, there is shown another exemplary apparatus 6000 for producing and delivering a vapour medicament 25. Apparatus 6000 may comprise one or more components already described above and description of such components has not been repeated below. FIG. 8B shows apparatus 5000 may comprise a pressurized reservoir 25, for example a cylinder, comprising both gas 15 and liquid medicament 17, and which may be in communication with chamber 12 via inlet 27. Apparatus 6000 may also comprise activation mechanism 27A. Apparatus 6000 may comprise liquid-absorbing member 43 disposed inside of chamber 12 between inlet 27 and vapour outlet 22.

The passing of gas 15 and liquid medicament 17 through liquid-absorbing member 43 and also the expansion of gas 15 together with liquid medicament 17 inside of chamber 12 upon release from pressurized reservoir 25 comprising both gas 15 and liquid medicament 17, may cause the production of vapour medicament 26 inside of chamber 12. Vapour medicament 26 and any residual gas 15 may continue to be delivered from the chamber 12 via vapour outlet 22.

Referring now to FIG. 9, there is disclosed a flowchart of an exemplary method 900 for producing and delivering vapour medicament 28. Method 900 and/or parts thereof may be performed using apparatus such as those disclosed herein. Method 900 and/or parts thereof may also be performed in conjunction with other methods disclosed herein. Method 900 may comprise: receiving gas 15 from gas reservoir 14 (see block 902); permitting expansion of gas 15 in the presence of liquid medicament 17 to produce vapour medicament 26 (see block 904); delivering at least some vapour medicament 26 through vapour outlet 22 while hindering delivery of at least some liquid medicament 17 through vapour outlet 22 (see block 906).

As explained above, the hindering of liquid medicament 17 from being delivered may be achieve using barrier 41 by filtering out droplets of liquid medicament 17 from a stream containing vapour medicament 26.

Referring now to FIG. 10, there is shown a flowchart of an exemplary method 1000 for producing and delivering vapour medicament 26. Method 1000 and/or parts thereof may be performed using apparatus such as those disclosed herein. Method 1000 and/or parts thereof may also be performed in conjunction with other methods disclosed herein. Method 1000 may comprise: receiving gas 15 from gas reservoir 14 (see block 1002); permitting expansion of gas 15 in the presence of liquid medicament 17 to produce vapour medicament 26 (see block 804); retaining at least some of liquid medicament 17 in liquid-absorbing member 43 while permitting passage of at least some of gas 15 through liquid-absorbing member 43 (see block 1006); delivering at least some of vapour medicament 26 through vapour outlet 22 (see block 1008).

As explained above, liquid-absorbing member 43 may comprise a sponge that may be absorb some of liquid medicament 17 and that is at least partially permeable to gas 15.

Referring now to FIG. 11, there is shown a flowchart of an exemplary method 1100 for producing and delivering vapour medicament 26. Method 1100 and/or parts thereof may be performed using apparatus such as those disclosed herein. Method 1100 and/or parts thereof may also be performed in conjunction with other methods disclosed herein. Method 1100 may comprise: receiving gas 15 via gas inlet 18 from gas reservoir 14 (see block 1102), receiving liquid medicament 17 from a liquid inlet 20, the liquid inlet 20 being separate from the gas inlet 18 (see block 1104); permitting expansion of the gas 15 in the presence of the liquid medicament 17 to produce vapour medicament 25 (see block 1106); delivering at least some of vapour medicament 26 through vapour outlet 22 (see block 1108).

The methods 900, 1000 and 1100 disclosed herein may comprise fewer steps or additional steps than those shown herein.

The above description is meant to be exemplary only, and one skilled in the relevant arts will recognize that changes may be made to the embodiments described without departing from the scope of the invention disclosed. For example, the blocks and/or operations in the flowcharts and drawings described herein are for purposes of example only. There may be many variations to these blocks and/or operations without departing from the teachings of the present disclosure. For instance, the blocks may be performed in a differing order, or blocks may be added, deleted, or modified. The present disclosure may be embodied in other specific forms without departing from the subject matter of the claims. Also, one skilled in the relevant arts will appreciate that while the components, apparatus and methods disclosed and shown herein may comprise a specific number of elements/components, the components, apparatus and methods could be modified to include additional or fewer of such elements/components. The present disclosure is also intended to cover and embrace all suitable changes in technology. Modifications which fail within the scope of the present invention will be apparent to those skilled in the art, in light of a review of this disclosure, and such modifications are intended to fail within the appended claims.

What is claimed is:

1. An apparatus for producing and delivering a vapour medicament, the apparatus comprising:
a gas inlet for receiving a gas from a source of pressurized gas;
a chamber for receiving a liquid medicament therein, the chamber being in communication with the gas inlet for permitting entrance and expansion of the gas in the chamber in the presence of the liquid medicament to produce the vapour medicament;
a vapour outlet in communication with the chamber for delivering the vapour medicament; and
a barrier disposed upstream of the vapour outlet and configured to hinder passage of at least some of the liquid medicament through the vapour outlet and permit passage of at least some of the vapour medicament through the vapour outlet.

2. The apparatus as defined in claim 1, wherein the barrier comprises an electrostatic surface that partially obstructs a flow path to the vapour outlet.

3. The apparatus as defined in claim 1, wherein the gas inlet and the vapour outlet are substantially coaxial.

4 The apparatus as defined in claim 1, wherein the chamber comprises a liquid inlet for receiving the liquid medicament from a liquid reservoir, the liquid inlet being separate from the gas inlet.

5. The apparatus as defined in claim 4, wherein the liquid reservoir comprises an outer wall containing the liquid medicament, the outer wall comprising a frangible portion having a reduced wall thickness relative to the wall thickness of another portion of the outer wall.

6. The apparatus as defined in claim 4, wherein the liquid inlet is oriented substantially transversely to the gas inlet.

7. The apparatus as defined in claim 1, comprising a first activation mechanism for causing the liquid medicament to enter the chamber and a second activation mechanism for causing the pressurized gas to enter the chamber, the first activation mechanism and the second activation mechanism being actuatable independently of each other.

8. The apparatus as defined in claim 1, comprising liquid medicament pre-stored in the chamber.

9. The apparatus as defined in claim 1, wherein the liquid medicament comprises a bronchial relaxant.

10. The apparatus as defined in claim 1, wherein the liquid medicament comprises a perfluorocarbon.

11. The apparatus as defined in claim 10, wherein the perfluorocarbon is selected from the group consisting of perfuoro-alkanes, perfluoroethers, perfluoro amines, perfluorodecalin, perfluorohexane, octafluoropropane, perfluoroperhydrophenanthrene, perfluorobutane, perfluorooctane, perefluoromethyldecalin, perfluorocarbons containing bromide, perfluorooctylbromide, perfluorodecalin, perfluorooctylethane, bis(perfluorobutyl)ethane, FC-43, FC-40, FC-5312, FC-77, FC-75, Rimar 101 and Caroxin.

12. The apparatus as defined in claim 10, wherein the perfluorocarbon is selected from the group consisting of FC-43, FC-77, perfluorodecalin, and perfluorooctylbromide.

13. The apparatus as defined in claim 1, comprising a receptacle for receiving a pressurized gas reservoir for coupling to the gas inlet, the receptacle having a longitudinal axis, the vapour outlet being disposed along the longitudinal axis of the receptacle.

14. The apparatus as defined in claim 13, comprising a first body defining at least part of the chamber and a second body defining at least part of the receptacle, the first body and second body configured to be threadably engaged to each other.

15. The apparatus as defined, in claim 1, comprising a liquid-absorbing member disposed inside of the chamber between the gas inlet and the vapour outlet, the liquid-absorbing member being at least partially permeable to the gas.

16. The apparatus as defined in claim 15, wherein the liquid-absorbing member comprises a sponge.

17. A method for producing and delivering a vapour medicament, the method comprising:
receiving a gas from a source of pressurized gas;
permitting expansion of the gas in the presence of a liquid medicament to produce the vapour medicament;
delivering at least some of the vapour medicament through a vapour outlet while hindering delivery of at least some of the liquid medicament through the vapour outlet.

18. The method as defined in claim 17, wherein hindering delivery of at least some of the liquid medicament comprises filtering out droplets of liquid medicament from a stream containing vapour medicament.

19. The method as defined in claim 17, wherein the liquid medicament comprises a bronchial relaxant.

20. The method as defined in claim 17, wherein the liquid medicament comprises a perfluorocarbon.

21. The method as defined in claim 20, wherein the perfluorocarbon is selected from the group consisting of perfluoro-alkanes, perfluoroethers, perfluoro amines, perfluorodecalin, perfluorohexane, octafluoropropane, perfluoroperhydrophenanthrene, perfluorobutane, perfluorooctane, perefluoromethyldecalin, perfluorocarbons containing bromide, perfluorooctylbromide, perfluorodecalin, perfluorooctylethane, bis(perfluorobutyl)ethane, FC-43, FC-40, FC-5312, FC-77, FC-75, Rimar 101 and Caroxin.

22. The method as defined in claim 20, wherein the perfluorocarbon is selected from the group consisting of FC-43, FC-77, perfluorodecalin and perfluorooctylbromide.

23. The method as defined in claim 17, wherein receiving the gas is preceded by receiving the liquid medicament.

24. An apparatus for producing and delivering a vapour medicament, the apparatus comprising:
a gas inlet for receiving a gas from a source of pressurized gas;
a chamber for receiving a liquid medicament therein, the chamber being in communication with the gas inlet for permitting entrance and expansion of the gas in the chamber in the presence of the liquid medicament to produce the vapour medicament;
a vapour outlet in communication with the chamber for delivering the vapour medicament; and
a liquid-absorbing member disposed inside of the chamber in a flow path between the gas inlet and the vapour outlet, the liquid-absorbing member being at least partially permeable to the gas.

25. The apparatus as defined in claim 24, wherein the liquid-absorbing member comprises a sponge.

26. The apparatus as defined in claim 24, wherein the gas inlet, and the vapour outlet are substantially coaxial.

27. The apparatus as defined in claim 24, wherein the chamber comprises a liquid inlet for receiving the liquid medicament from a liquid reservoir, the liquid inlet being separate from the gas inlet.

28. The apparatus as defined in claim 27, wherein the liquid inlet is oriented substantially transversely to the gas inlet.

29. The apparatus as defined in claim 24, comprising a first activation mechanism for causing the liquid medicament to enter the chamber and a second activation mechanism for causing the pressurized gas to enter the chamber, the first activation mechanism and the second activation mechanism being actuatable independently of each other.

30. The apparatus as defined in claim 24, comprising liquid medicament pre-stored in the chamber.

31. The apparatus as defined in claim 27, wherein the liquid reservoir comprises an outer wall containing the liquid medicament, the outer wall comprising a frangible portion having a reduced wall thickness relative to the wall thickness of another portion of the outer wall.

32. The apparatus as defined in claim 24, wherein the liquid medicament comprises a bronchial relaxant.

33. The apparatus as defined in claim 24, wherein the liquid medicament comprises a perfluorocarbon.

34. The apparatus as defined in claim 33, wherein the perfluorocarbon is selected from the group consisting of perfluoro-alkanes, perfluoroethers, perfluoro amines, perfluorodecalin, perfluorohexane, octafluoropropane, perfluoroperhydrophenanthrene, perfluorobutane, perfluorooctane, perefluoromethyldecalin, perfluorocarbons containing bromide, perfluorooctylbromide, perfluorodecalin, perfluorooctylethane, bis(perfluorobutyl)ethane, FC-43, FC-40, FC-S312, FC-77, FC-75, Rimar 101 and Caroxin.

35. The apparatus as defined in claim 33, wherein the perfluorocarbon is selected from the group consisting of FC-43, FC-77, perfluorodecalin, and perfluorooctylbromide.

38. The apparatus as defined in claim 24, comprising a receptacle for receiving a pressurized gas reservoir for coupling to the gas inlet, the receptacle having a longitudinal axis, the vapour outlet being disposed along the longitudinal axis of the receptacle.

37. The apparatus as defined in claim 36, comprising a first body defining at least part of the chamber and a second body defining at least part of the receptacle, the first body and second body configured to be threadably engaged to each other.

38. A method for producing and delivering a vapour medicament, the method comprising:
receiving a gas from a source of pressurized gas;
permitting expansion of the gas in the presence of a liquid medicament to produce the vapour medicament;
retaining at least some of the liquid medicament in a liquid-absorbing member while permitting passage of at least some of the gas through the liquid-absorbing member; and
delivering at least some of the vapour medicament through a vapour outlet.

39. The method as defined in claim 38, wherein delivering at least some of the vapour further comprising hindering delivery of at least some of the liquid-medicament by filtering out droplets of liquid medicament from a stream containing vapour medicament.

40. The method as defined in claim 38, wherein the liquid medicament comprises a bronchial relaxant.

41. The method as defined in claim 38, wherein the liquid medicament comprises a perfluorocarbon.

42. The method as defined in claim 41, wherein the perfluorocarbon is selected from the group consisting of perfluoro-alkanes, perfluoroethers, perfluoro amines, perfluorodecalin, perfluorohexane, octafluoropropane, perfluoroperhydrophenanthrene, perfluorobutane, perfluorooctane, perefluoromethyldecalin, perfluorocarbons containing bromide, perfluorooctylbromide, perfluorodecalin, perfluorooctylethane, bis(perfluorobutyl)ethane, FC-43, FC-40, FC-5312, FC-77, FC-75, Rimar 101 and Caroxin.

43. The method as defined in claim 41, wherein the perfluorocarbon is selected from the group consisting of FC-43, FC-77, perfluorodecalin, and perfluorooctylbromide.

44. The method as defined in claim 38, wherein receiving the liquid medicament in the liquid-absorbing member precedes receiving the gas from the source of pressurized gas.

45. An apparatus for producing and delivering a vapour medicament, the apparatus comprising:
a chamber comprising a gas inlet for receiving a gas from a source of pressurized gas and a liquid inlet for receiving a liquid medicament from a liquid reservoir, the gas inlet and the liquid inlet being separate from each other, the chamber permitting expansion of the gas in the chamber in the presence of the liquid medicament to produce the vapour medicament; and
a vapour outlet in communication with the chamber tor delivering the vapour medicament.

46. The apparatus as defined in claim 45, wherein the apparatus comprises a barrier disposed upstream of the vapour outlet and configured to hinder passage of at least some of the liquid medicament through the vapour outlet and permit passage of at least some of the vapour medicament through the vapour outlet.

47. The apparatus as defined in claim 45, wherein the gas inlet and the vapour outlet are substantially coaxial.

48. The apparatus as defined in claim 45, wherein the liquid inlet is oriented substantially transversely to the gas inlet.

49. The apparatus as defined in claim 45, comprising a first activation mechanism for causing the liquid medicament to enter the chamber and a second activation mechanism for causing the pressurized gas to enter the chamber, the first activation mechanism and the second activation mechanism being actuatable independently of each other.

50. The apparatus as defined in claim 45, wherein the liquid medicament comprises a bronchial relaxant.

51. The apparatus as defined in claim 45, wherein the liquid medicament comprises a perfluorocarbon.

52. The apparatus as defined in claim 51, wherein the perfluorocarbon is selected from the group consisting of perfluoro-alkanes, perfluoroethers, perfluoro amines, perfluorodecalin, perfluorohexane, octafluoropropane, perfluoroperhydrophenanthrene, perfluorobutane, perfluorooctane, perefluoromethyldecalin, perfluorocarbons containing bromide, perfluorooctylbromide, perfluorodecalin, perfluorooctylethane. bis(perfluorobutyl)ethane, FC-43, FC-40, FC-5312, FC-77, FC-75, Rimar 101 and Caroxin.

53. The apparatus as defined in claim 51, wherein the perfluorocarbon is selected from the group consisting of FC-43, FC-77, perfluorodecalin, and perfluorooctylbromide.

54. The apparatus as defined in claim 45, comprising a receptacle for receiving a pressurized gas reservoir for coupling to the gas inlet, the receptacle having a longitudinal axis, the vapour outlet being disposed along the longitudinal axis of the receptacle.

55. The apparatus as defined in claim 54, comprising a first body defining at least part of the chamber and a second body defining at least part of the receptacle, the first body and second body configured to be threadably engaged to each other.

56. The apparatus as defined in claim 45, comprising a liquid-absorbing member disposed inside of the chamber between the gas inlet and the vapour outlet, the liquid-absorbing member being at least partially permeable to the gas.

57. The apparatus as defined in claim 56, wherein the liquid-absorbing member comprises a sponge.

58. The apparatus as defined in claim 45, wherein the liquid reservoir comprises an outer wall containing the liquid medicament, the outer wall comprising a frangible portion having a reduced wall thickness relative to the wall thickness of another portion of the outer wall.

59. A method for producing and delivering a vapour medicament, the method comprising:
receiving a liquid medicament into a chamber via a liquid inlet;
receiving a gas into the chamber via a gas inlet from a source of pressurized gas, the liquid inlet being separate from the gas inlet;
in the chamber, permitting expansion of the gas in the presence of the liquid medicament to produce the vapour medicament; and
delivering at least some of the vapour medicament through a vapour outlet.

60. The method as defined in claim 59, wherein delivering at least some of the vapour further comprises hindering delivery of at least some of the liquid medicament by filtering out droplets of liquid medicament from a stream containing vapour medicament.

61. The method as defined in claim 59, wherein the liquid medicament comprises a bronchial relaxant.

62. The method as defined in claim 59, wherein the liquid medicament comprises a perfluorocarbon.

63. The method as defined in claim 62, wherein the perfluorocarbon is selected from the group consisting of perfluoro-alkanes, perfluoroethers, perfluoro amines, perfluorodecalin, perfluorohexane, octafluoropropane, perfluoroperhydrophenanthrene, perfluorobutane, perfluorooctane, perefluoromethyldecalin, perfluorocarbons containing bromide, perfluorooctylbromide, perfluorodecalin, perfluorooctylethane, bis(perfluorobutyl)ethane, FC-43, FC-40, FC-5312, FC-77, FC-75, Rimar 101 and Caroxin.

64. The method as defined in claim 62, wherein the perfluorocarbon is selected from the group consisting of FC-43, FC-77, perfluorodecalin, and perfluorooctylbromide.

65. The method as defined in claim 59, wherein receiving the liquid medicament from the liquid inlet precedes receiving the gas from the source of pressurized gas.

66. An apparatus for producing and delivering a vapour medicament, the apparatus comprising:
a gas inlet for receiving a gas from a pressurized gas reservoir;
a chamber for receiving a liquid medicament therein, the chamber being in communication with the gas inlet for permitting entrance and expansion of the gas in the chamber in the presence of the liquid medicament to produce the vapour medicament;
a vapour outlet in communication with the chamber for delivering the vapour medicament; and
a receptacle for receiving the pressurized gas reservoir, the receptacle having a longitudinal axis and the vapour outlet being disposed along the longitudinal axis of the receptacle.

67. The apparatus as defined in claim 66, wherein the apparatus comprises a barrier disposed upstream of the vapour outlet and configured to hinder passage of at least some of the liquid medicament through the vapour outlet and permit passage of at least some of the vapour medicament through the vapour outlet.

68. The apparatus as defined in claim 67, wherein the barrier comprises an electrostatic surface that partially obstructs a flow path to the vapour outlet.

69. The apparatus as defined in claim 66, wherein the gas inlet and the vapour outlet are substantially coaxial.

70. The apparatus as defined in claim 86, wherein the chamber comprises a liquid inlet for receiving the liquid medicament from a liquid reservoir, the liquid inlet being separate from the gas inlet.

71. The apparatus as defined in claim 70, wherein the liquid inlet is oriented substantially transversely to the gas inlet.

72. The apparatus as defined in claim 66, comprising a first activation mechanism for causing the liquid medicament to enter the chamber and a second activation mechanism for causing the pressurized gas to enter the chamber, the first activation mechanism and the second activation mechanism being actuatable independently of each other.

73. The apparatus as defined in claim 66, comprising liquid medicament pre-stored in the chamber.

74. The apparatus as defined in claim 68, wherein the liquid reservoir comprises an outer wall containing the liquid medicament, the outer wall comprising a frangible portion having a reduced wall thickness relative to the wall thickness of another portion of the outer wall.

75. The apparatus as defined in claim 66, wherein the liquid medicament comprises a bronchial relaxant.

76. The apparatus as defined in claim 66, wherein the liquid medicament comprises a perfluorocarbon.

77. The apparatus as defined in claim 76, wherein the perfluorocarbon is selected from the group consisting of perfluoro-alkanes, perfluoroethers, perfluoro amines, perfluorodecalin, perfluorohexane, octafluoropropane, perfluoroperhydrophenanthrene, perfluorobutane, perfluorooctane, perefluoromethyldecalin, perfluorocarbons containing bromide, perfluorooctylbromide, perfluorodecalin, perfluorooctylethane, bis(perfluorobutyl)ethane, FC-43, FC-40, FC-5312, FC-77, FC-75, Rimar 101 and Caroxin.

78. The apparatus as defined in claim 76, wherein the perfluorocarbon is selected from the group consisting of FC-43, FC-77, perfluorodecalin, and perfluorooctylbromide.

79. The apparatus as defined in claim 66, comprising a first body defining at least part of the chamber and a second body defining at least pail of the receptacle, the first body and second body configured to be threadably engaged to each other.

80. An apparatus for producing and delivering a vapour medicament, the apparatus comprising:
a storage reservoir containing both a liquid medicament and a pressurized gas;
a chamber comprising an inlet for receiving the liquid medicament and the gas from the storage reservoir, the chamber permitting expansion of the gas in the chamber in the presence of the liquid medicament to produce the vapour medicament; and
a vapour outlet in communication with the chamber for delivering the vapour medicament.

81. The apparatus as defined claim 80, further comprising a receptacle for receiving the reservoir, the receptacle having a longitudinal axis and the vapour outlet being disposed along the longitudinal axis of the receptacle.

82. The apparatus as defined in claim 81, comprising a first body defining at least part of the chamber and a second body defining at least part of the receptacle, the first body and second body configured to be threadably engaged to each other.

83. The apparatus as defined in claim 80, further comprising a barrier disposed upstream of the vapour outlet and configured to hinder passage of at least some of the liquid medicament through the vapour outlet and permit passage of at least some of the vapour medicament through the vapour outlet.

84. The apparatus as defined in claim 80, further comprising a liquid-absorbing member disposed inside of the chamber in a flow path between the inlet and the vapour outlet, the liquid-absorbing member being at least partially permeable to the gas.

85. The apparatus as defined in claim 80, comprising an activation mechanism for causing the gas and the liquid medicament to enter the chamber.

86. The apparatus as defined in claim 80, wherein the liquid medicament comprises a bronchial relaxant.

87. The apparatus as defined in claim 80, wherein the liquid medicament comprises a perfluorocarbon.

88. The apparatus as defined in claim 87, wherein the perfluorocarbon is selected from the group consisting of perfluoro-alkanes, perfluoroethers, perfluoro amines, perfluorodecalin, perfluorohexane, octafluoropropane, perfluoroperhydrophenanthrene, perfluorobutane, perfluorooctane, perefluoromethyldecalin, perfluorocarbons containing bromide, perfluorooctylbromide, perfluorodecalin, perfluorooctylethane, bis(perfluorobutyl)ethane, FC-43, FC-40, FC-5312, FC-77, FC-75, Rimar 101 and Caroxin.

89. The apparatus as defined in claim 87, wherein the perfluorocarbon is selected from the group consisting of FC-43, FC-77, perfluorodecalin, and perfluorooctylbromide.

90. A liquid medicament reservoir for use in an apparatus for producing and delivering a vapour medicament, the liquid medicament reservoir comprising:
a quantity of liquid medicament; and
an outer wall containing the liquid medicament, the outer wall comprising a frangible portion having a reduced wall thickness relative to the wall thickness of another portion of the outer wall.

91. The liquid medicament reservoir as defined in claim 30, wherein the reduced wall thickness is up to about 25% of the wall thickness of the other portion of the outer wall.

92. The liquid medicament reservoir as defined in claim 90, wherein the reduced wall thickness is up to about 50% of the wall thickness of the other portion of the outer wall.

93. The liquid medicament reservoir as defined in claim 90, wherein the reduced wall thickness is up to about 75% of the wall thickness of the other portion of the outer wall.
